(19) United States
(12) Patent Application Publication
(10) Pub. No.: US 2023/0050289 A1
(43) Pub. Date: Feb. 16, 2023

(54) Medical Diagnostic Kit

(71) Applicant: T-Mobile Innovations LLC, Overland Park, KS (US)

(72) Inventors: Julia Lea GRONEWOLD, Wichita, KS (US); Joao TEIXEIRA, Shawnee, KS (US)

(21) Appl. No.: 17/978,020

(22) Filed: Oct. 31, 2022

Related U.S. Application Data
(62) Division of application No. 29/779,226, filed on Apr. 16, 2021.

(51) Int. Cl. A61B 5/00; G16H 40/67

(52) U.S. Cl. CPC A61B 5/0022; G16H 40/67

(57) ABSTRACT

Illustrated and claimed herein is the decorative design for a medical diagnostic kit. The medical diagnostic kit may be used for telemedicine. The medical diagnostic kit may comprise a plurality of components including a first component, a second component, a third component, and/or a fourth component. One of the components such as the first component may comprise one or more of a camera, a holographic projector, or a pulse reader. The other components may comprise a blood glucose monitor, an ultrasound, and a dermascope. The second, third, and fourth components may be detachable from the first component as well as from each other. The medical diagnostic kit may comprise a color gradation from the first component to the fourth component with the first component being the lightest color and the fourth component being the darkest color. Upon allowance of this application, the appendices may be deleted from the application text, to only remain a part of this file, and need not be printed as part of such patent as may issue (since it does not relate to claimed matter).

### CROSS-REFERENCE TO RELATED APPLICATIONS

This application is a divisional of and claims priority under 35 U.S.C. § 120 to U.S. patent application Ser. No. 29/779,226, filed on Apr. 16, 2021, entitled “Medical Diagnostic Kit,” by Julia Lea Gronewold, et al., which is incorporated herein by reference in its entirety for all purposes.

### STATEMENT REGARDING FEDERALLY SPONSORED RESEARCH OR DEVELOPMENT

Not applicable.

### DESCRIPTION

Be it known that we, Julia Lea Gronewold and Joao Teixeira, have invented a new, original, and ornamental design for a medical diagnostic kit or similar article. The accompanying drawings forming a part of the specification are described as follows:

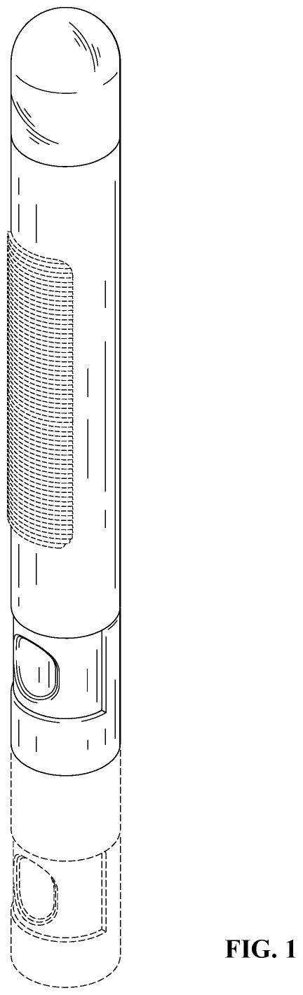

FIG. 1 is a front perspective view of a medical diagnostic kit in accordance with our new design;

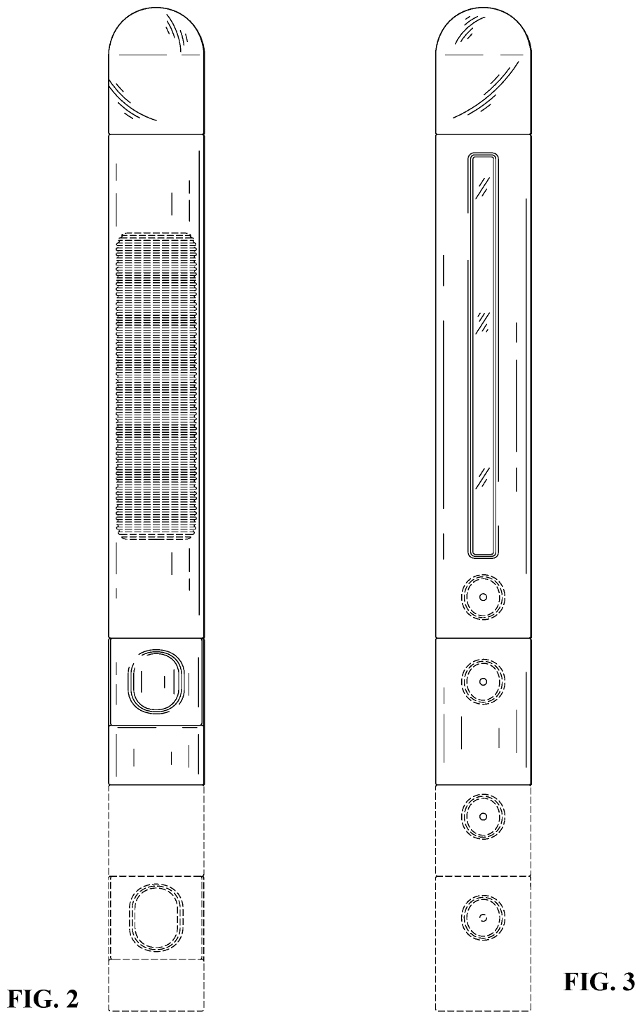

FIG. 2 is a front elevation view thereof;

FIG. 3 is a rear elevation view thereof;

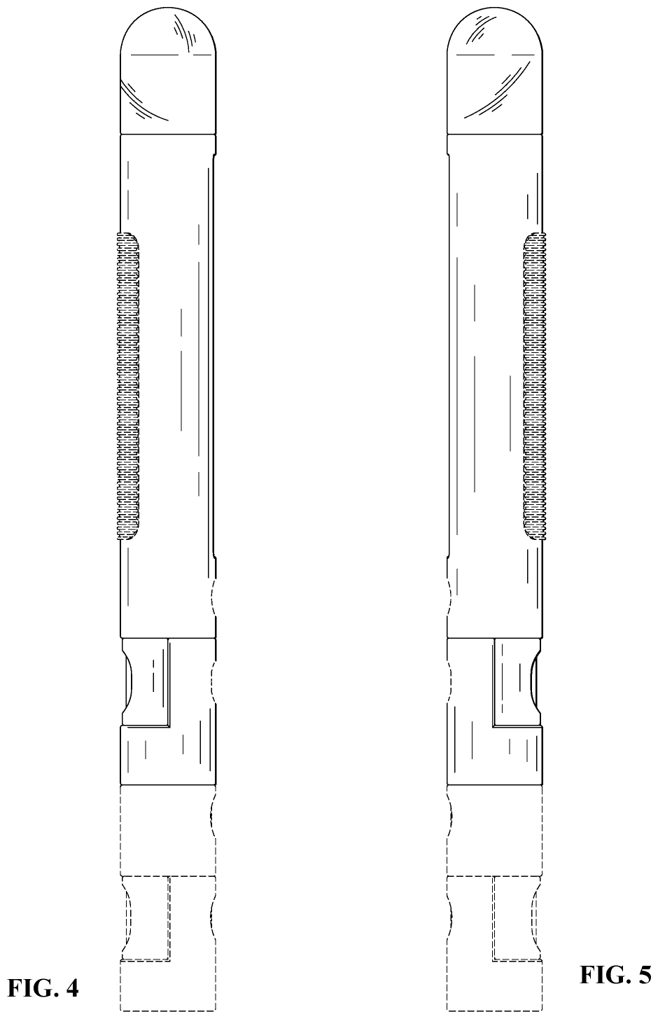

FIG. 4 is a side view thereof;

FIG. 5 is a side view thereof, for the side opposite that of FIG. 4;

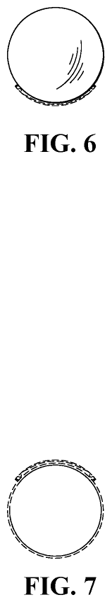

FIG. 6 is a top view thereof;

FIG. 7 is a bottom view thereof;

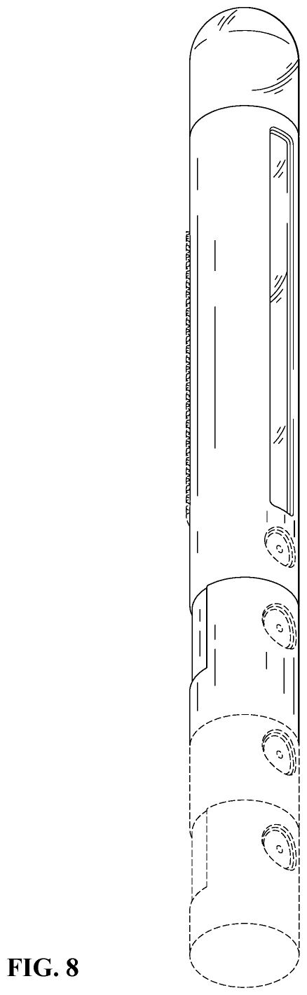

FIG. 8 is a rear perspective view thereof;

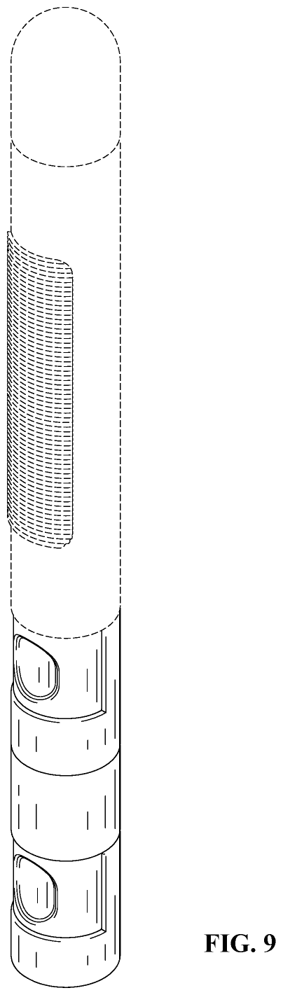

FIG. 9 is a front perspective view of another medical diagnostic kit in accordance with our new design;

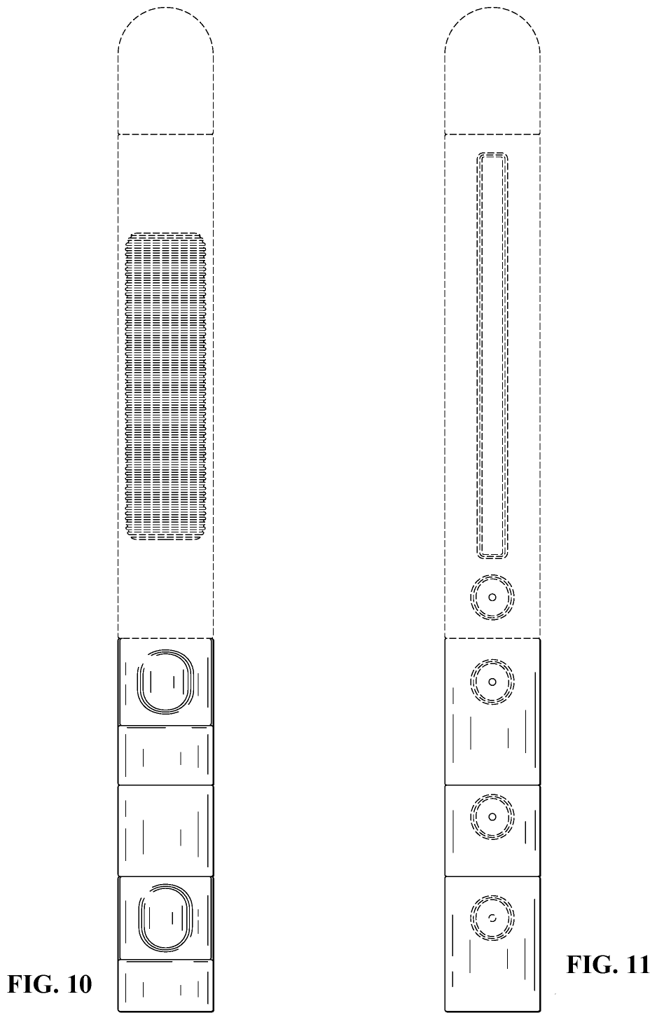

FIG. 10 is a front elevation view thereof;

FIG. 11 is a rear elevation view thereof;

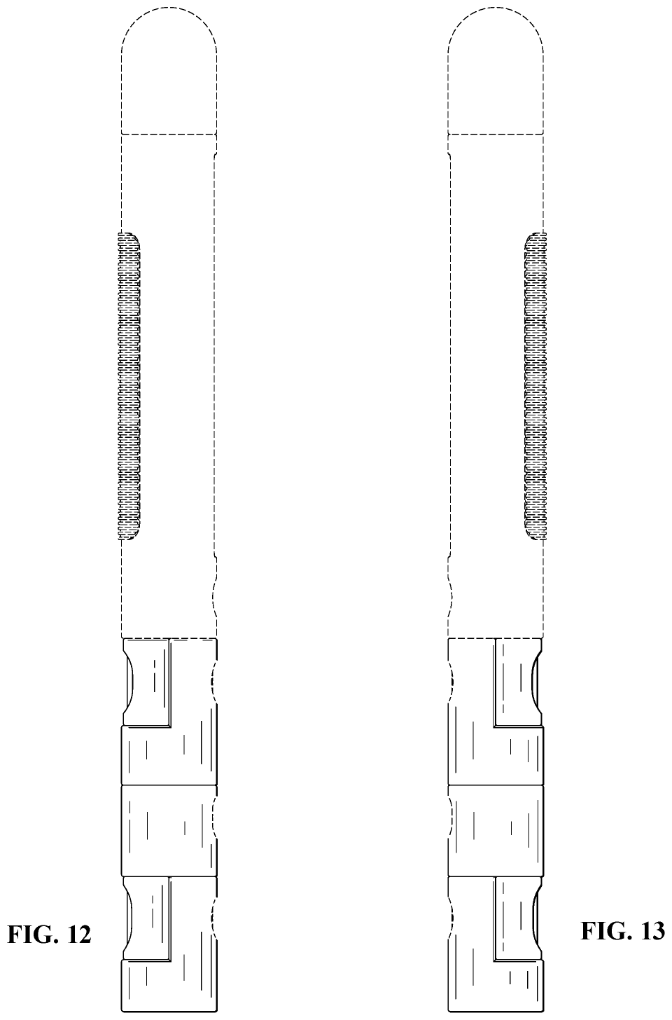

FIG. 12 is side view thereof;

FIG. 13 is a side view thereof, for the side opposite that of FIG. 12;

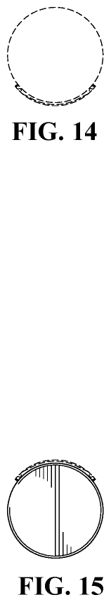

FIG. 14 is a top view thereof;

FIG. 15 is a bottom view thereof;

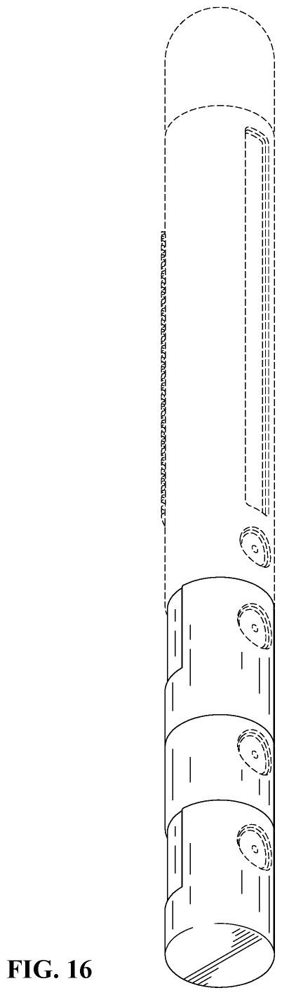

FIG. 16 is rear perspective view thereof;

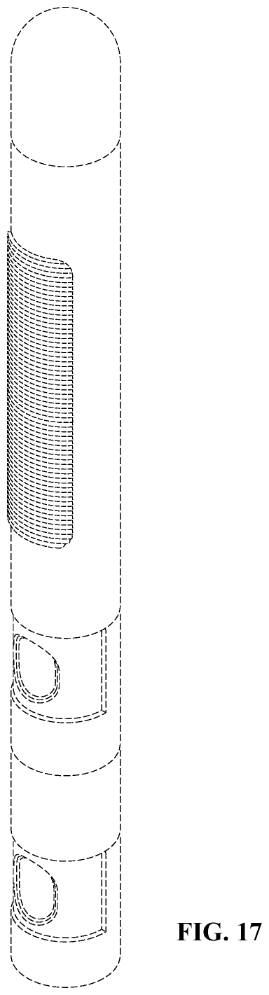

FIG. 17 is a front perspective view of another medical diagnostic kit in accordance with our new design;

FIG. 18 is a front elevation view thereof;

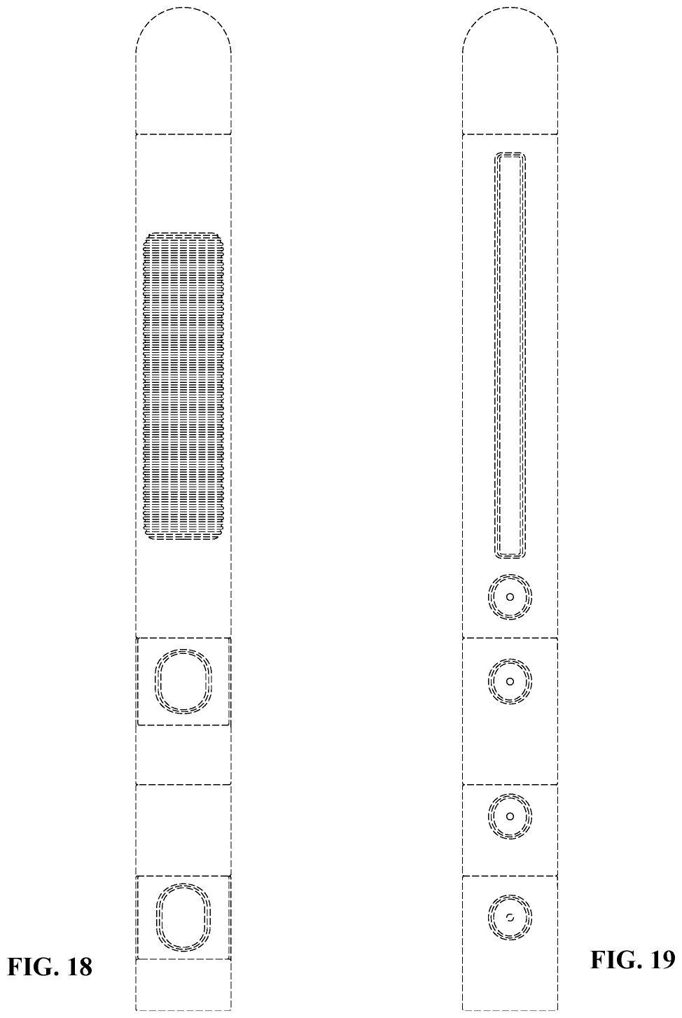

FIG. 19 is a rear elevation view thereof;

FIG. 20 is a side view thereof;

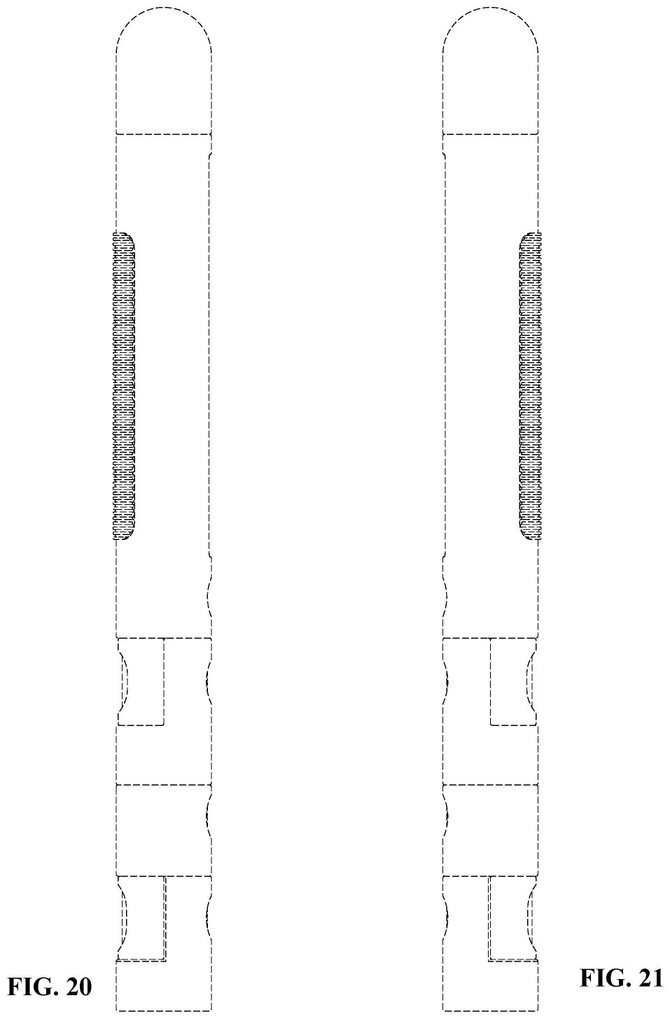

FIG. 21 is a side view thereof, for the side opposite that of FIG. 20;

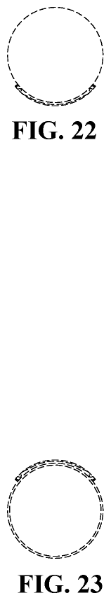

FIG. 22 is a top view thereof;

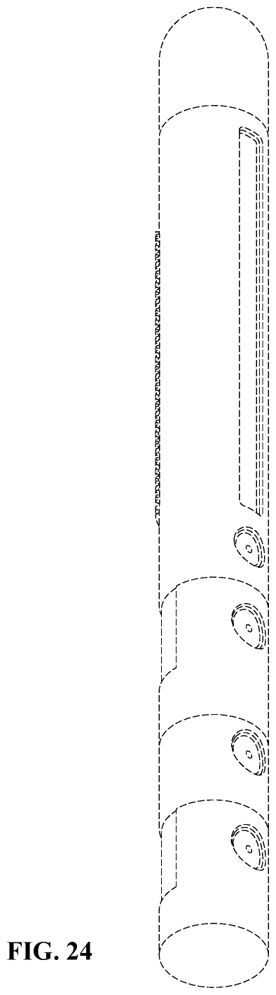

FIG. 23 is a bottom view thereof; and

FIG. 24 is a rear perspective view thereof.

The broken lines shown in the drawings illustrate portions of the medical diagnostic kit that form no part of the claimed design.

1. We claim the ornamental design of a medical diagnostic kit, as shown.
